(19) United States
(12) Patent Application Publication
(10) Pub. No.: US 2016/0326493 A1
(43) Pub. Date: Nov. 10, 2016

(54) MULTILINEAGE STEM CELLS DERIVED FROM THE PERIPHERAL BLOOD AND USES THEREOF

(71) Applicant: Vivex Biomedical, Inc., Marietta, GA (US)

(72) Inventors: Theodore I. Malinin, Key Biscayne, FL (US); Dorothy R. Malinin, Key Biscayne, FL (US); Wendy W. Weston, Miami, FL (US)

(73) Assignee: Vivex Biomedical, Inc., Marietta, GA (US)

(21) Appl. No.: 14/706,165

(22) Filed: May 7, 2015

(51) Int. Cl. C12N 5/077; A61K 35/30; A61K 35/32; C12N 5/0793; A61K 35/28

(52) U.S. Cl. CPC C12N 5/0655; C12N 5/0654; C12N 5/0619; A61K 35/28; A61K 35/32; A61K 35/30; C12N 2501/15; C12N 2500/38; C12N 2501/04; C12N 2500/30; C12N 2501/33; C12N 2500/24; C12N 2500/34; C12N 2506/11; A61K 2035/124

(57) ABSTRACT

The present invention discloses recovery and isolation of mesenchymal stem cells from the peripheral blood. This has an advantage of being able to obtain multilineage inducible cells from readily obtainable and autologous allogenic blood. With the present invention it is no longer necessary to depend on cadaver donor bone marrow, a technically complicated and an expensive process. Instead the patient may obtain the needed stem cells from a relatively small amount of his/her own blood, or from readily available blood donors.

### TECHNICAL FIELD

This invention is related to multilineage stem cells derived from the peripheral blood and uses thereof.

### BACKGROUND OF THE INVENTION

Mesenchymal stem cells (MSCs) also known as bone marrow stromal cells, fibroblastic colony forming units or multilineage inducible cells, have been recovered from the bone marrow by several investigators (de Camargo Bittencourt et al, 2006; Schiller and D'Ippolito, 2010; D'Ippolito et al 1999). These bone marrow derived cells, have been described first by Friedenstein et al (1974) who found that MSCs adhere to culture plates, look like in vitro fibroblast, and build up colonies. Since then many investigators have isolated MSCs and controlled their differentiation in vitro into cartilaginous, and other osseous tissues using, among other things, growth factors with an objective of this new technology for regenerating mesenchymal tissue.

The present invention is based on an earlier finding of bone marrow recovery in lethally irradiated animals by application of peripheral blood white cells. (Malinin et al 1965). Post-natal multilineage stem cells usually derived from bone marrow have been previously described. These cells have the ability to renew and differentiate themselves into multilineages of connective tissues including bone, cartilage, muscle, adipose tissue, tendons and bone marrow stroma. The multilineage cells have great potential for the treatment of human diseases. Evidence of stem cells in the peripheral blood of mice was previously presented by Goodman and Hodson (1962).

Mesenchymal stem cells are undifferentiated self-renewable cells that can be serially propagated, and can be made to differentiate into mature cells and tissues with specialized functions, such as cartilage and bone. MSCs can be made to differentiate virtually in all other tissues as well. MSCs have been used and can be used to regenerate damaged cartilage, bone and other tissues. Long term renewal of these cells has been achieved in culture. The same attributes are present in embryonic cells, but the use of human embryonic stem cells is fraught with difficulties because of moral, ethical and legal concerns regarding early embryos. This limitation has stimulated scientists to find substitutes for embryonic stem cells among post-natal tissues. The present invention reveals the method of finding and isolating MSCs from easily accessible and abundant tissue, blood. The cells in question are present in blood in very small quantities. The method of their isolation in cell culture is unique and results in a cell composition which no longer constitutes unaltered blood.

These and other benefits of the present invention and the method of preparing it are described hereinafter.

### SUMMARY OF THE INVENTION

Conditions for in vitro isolation of post-natal multilineage inducible cells (MIC) from blood are disclosed herein. In certain embodiments, such culture conditions may include length of incubation, oxygen and CO₂ atmosphere content, extracellular matrix content, growth factors, vitamin and nutritional supplements, cell densities or any combination thereof. The disclosed MIC cells have a unique molecular profile. For example, in one embodiment the cells express CD90 and low levels of CD45, markers indicating a variety of stem cells, including human mesenchymal stromal cells markers. Markers for antigen producing cells as well as hematopoietic cells are absent. MIC cells can be maintained in culture without detectable change in their characteristic molecular profile. Such in vitro MIC cell populations have multi germ layer differentiation potential and can be differentiated into definitive tissue phenotype.

In vitro condition for isolation of post-natal multilineage inducible cells from peripheral blood (MICPB) are disclosed herein. In some embodiments the in vitro conditions described cell culture environment, including for example fibronectin coated flasks, oxygen tension, cell density, various incubation times, growth factors, vitamins or a combination of the above. The disclosed MICPB cells have unique molecular profiles and morphologic appearance. For example, in one embodiment, MICPB express high levels of CD90 maker and nominal levels of CD45 marker. CD90 is commonly used as mesenchymal stem cell marker. CD45 is hematopoietic progenitor marker and is also found in low levels on mesenchymal stem cells. Significantly there was no HLADR marker which is an immune cells marker. Other markers (CD73, CD29, CD105, SSEA4, CD34) were not present. The lack of CD34 and HLADR and positive expression of CD90 indicates that MICPB cells are indeed mesenchymal stem cells. Such a mesenchymal stem cell population has a multi germ layer differentiation potential and therefore can be used in a treatment of a number of medical conditions in which cell repopulation and repair might be beneficial. An example would be repair of articular cartilage defects or bone defects.

### DEFINITIONS

Unless otherwise noted technical terms are used in accordance with accepted conventions. Definitions of common terms in molecular biology can be found in Genes V. by Benjamin Lewis. Oxford University Press, 1994 (ISBN 0-19-8542798-9) or in H. Kreuzer and A. Massey Molecular biology and Biotechnology: A guide for Teachers. 3rd Ed. Am Society for Microbiology, 2006 (ISBN: 9781555814717/1555814719).

Expand. A process by which number of cells in a cell culture vessel is increased due to cell division, but without changing the character of the cells.

Induce. To cause to move forward to a definite type. As an example, certain cell types can be made to become specialized, from a previously cell specialized cell type. The cells can be induced to differentiate.

Isolated. An isolated cell is a cell that had been separated from other cells and tissue components. Cells can be separated by several methods, including mechanical enzymatic dispersion. In blood cells can be isolated by creating low oxygen tension. Appropriate cell culture condition. Any condition in a culture incubator below normal atmospheric oxygen level, usually about 21%. In a particular embodiment low oxygen tension and below 21% to as low as 5 or 3% oxygen. Low oxygen conditions are used to approximate oxygen requirements by different types of cells. They are not the same as hypoxic conditions, as they are intended to mimic physiologic conditions. Hypoxia, on the other hand denotes less than normal physiologic conditions.

Marker. A protein or other molecule exposed of the cell surface which identifies a particular cell. Cell markers can be detected by immunocy to chemistry. Fluorescence activated cell sorting or an enzymatic analysis.

Multilineage-inducible cell. A cell that can differentiate into more than one cell type. A multilineage cell is capable of differentiating into cell types from more than a single germ layer, i.e. ectodermal, mesodermal or endodermal. In a particular embodiment a multilineage—inducible cell can differentiate into chondrocytes, osteoblasts, neurons and endocrine cells.

Post-natal. Meaning after birth. This includes a new born, a child, an adolescent or an adult, regardless of age.

MICPB Cells. Disclosed herein are isolated post-natal multilineage inducible cells derived from the peripheral blood.

### BRIEF DESCRIPTION OF THE DRAWINGS

The invention will be described by way of example and with reference to the accompanying drawings in which:

FIG. 1 shows a histogram obtained from gradient separated blood of a single donor, after 30 days incubation. The histogram shows the presence of cells with CD90 marker, which is an attribute of stem cells.

FIG. 2 shows a histogram of a gradient separated blood from a single donor after 30 days of incubation. The graph shows nominal levels of CD45 markers indicating the presence of mesenchymal stem cells as well as hematopoietic stem cells. This marker is also present in low levels in stem cells isolated from bone marrow.

FIG. 3 shows representative cells isolated from the peripheral blood after 14 days in tissue culture. Each cell has the morphologic appearance of primitive mesenchymal cells, similar to those isolated from bone marrow. Hoffman modulation microscopy ×100.

FIGS. 4A and 4B shows several elongated cells (4A) and a cluster of cells (4B) isolated from peripheral blood after 30 days incubation. These cells showed high levels of CD90 markers. Hoffman modulation microscopy images ×40.

FIG. 5 is a view of a pre-filled syringe with a predetermined amount of the mixture of the cells in a liquid, preferably saline or other suitable liquid.

FIG. 6 is a view of a sealed container or vial containing a mixture of the cells in saline or other suitable liquid or gel.

### DETAILED DESCRIPTION OF THE INVENTION

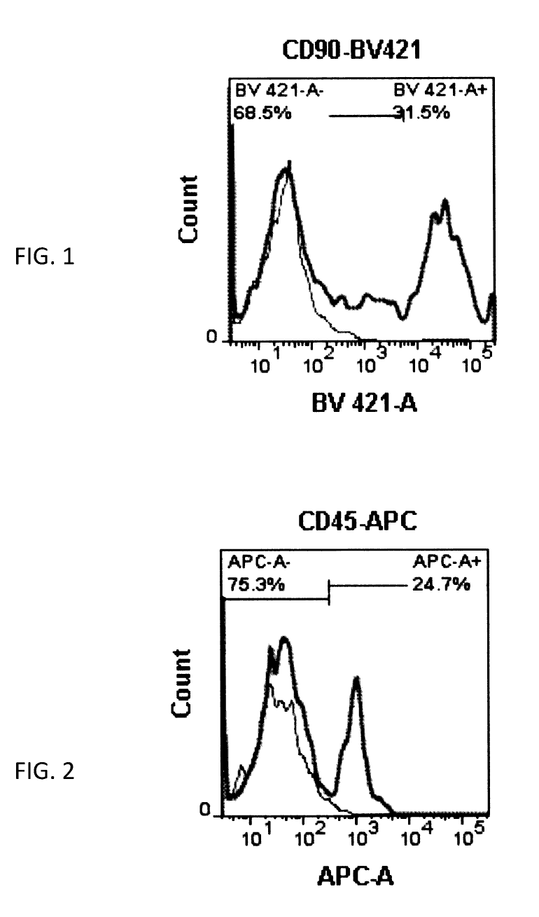

Disclosed herein are post-natal, multilineage inducible cells derived from the peripheral blood of a human. These cells, isolated in a specifically designed tissue culture environment, exhibit high levels of mesenchymal stem cell marker, CD90, as shown in FIG. 1, as well low levels of CD45 markers, as shown in FIG. 2. The cells are devoid of other markers including immune cell marker (HLADR). The cells have been isolated from peripheral blood of an adult human individual. This, to the best knowledge of the inventors, had not been done previously. The cells isolated from blood represent a very small number of the non-erythroid cell population, and have been isolated only by employing specifically designed cell cultures technique. The end result is a specific cell population composed of mesenchymal stem cells which had to be expanded. The population derived through segregation, isolation and expansion of individual cells into multi-lineage cell colonies is unique and in does not resemble the original tissue (blood) from which it has been derived.

Similar cells have been isolated from different tissues including cord blood, bone marrow and fat, but not from the human post-natal peripheral blood.

Methods of isolating multilineage -inducible cells are also disclosed. Such methods include obtaining blood through venipuncture and collecting it in quantities from 150 to 500 ml into containers to which 20,000 units or 40,000 units of heparin had been added. Aliquots of blood (15 ml) are placed over Ficol (15 ml) in centrifuge tubes (50 ml) and centrifuged at 400 g for 30 min. To obtain an adequate amount of cells at least 8 to 40 tubes are needed. Rings from each tube are collected and pooled into 50 ml conical tubes. The tubes are then filled with phosphate buffered saline (PBS), and centrifuged at 400 g. The supernatant fluid is then aspirated and the cell pellet is re-suspended in an expansion media. As incubation progresses the MICPB cells are self-selected.

When the cells reach a critical volume they can be either used fresh in a recipient or they can be cryopreserved for future use.

Also disclosed herein are methods for inducing MICPB cells to undergo chondrogenic differentiation. This is achieved by placing them into chondrogenic medium which can be either serum free or supplemented with sera of various kinds In some embodiments, it will comprise human serum, fetal bovine serum or calf serum, porcine embryonic or post-natal serum, etc. In other embodiments, the tissue culture media will be supplemented with extracts of tissues whose phenotype it is desirable to replicate. In another embodiment, microparticles of such tissue, fresh, frozen or desiccated are added to the medium to stimulate cells to differentiate into specific phenotypes. As an example, microparticulate cartilage can be added to the media to stimulate MICPB cells to engage into chondrogenic differentiation. In another embodiment, ascorbic acid, TG F-beta 3, dexamethasone, sodium pyruvate, proline, transferrin, selenous acid, and insulin are added to the medium as is done with multilineage inducible cells derived from bone marrow. In other embodiments, neural differentiation, osteogenic differentiation, adipogenic differentiation and other specific tissue differentiation as is done with multilineage inducible cells derived from bone marrow are disclosed.

Also disclosed herein are methods of treating disorders such as, but not limited to, cartilage disorder, bone disorder, a neurological disorder, liver disorder, kidney disorder, myocardial disorder, muscle disorder, in a subject by administering a therapeutically effective amount of disclosed multilineage inducible cells derived from the peripheral blood. Some methods further include inducing the cells to differentiate into chondral, osseous, neural, myogenic, tendineous and other tissue. In some methods the cells are placed locally into a subject. In other examples cells are administered systemically to a subject.

MICPB cells express at least the single, significant cell marker, CD90, as shown in FIG. 1. MICPB cells are relatively small cells with single pseudopodium and prominent granular cytoplasm and a small nucleus. Several cells may be clustered together. These cells have been so far isolated from fresh peripheral blood for the first time. MICPB cells may be isolated from blood by several methods known in the art or by combination thereof, including the methods disclosed herein. In one embodiment, MICPB cells are selected and expanded utilizing cell culture techniques. In another embodiment, MICPB cells are isolated from blood buffy coat on the basis of physical properties of these cells. Several techniques are known in the art by which MICPB cells may be isolated based on set of markers expressed by these cells including for example immobilized marker specific antibodies, magnetic activated cell sorting and fluorescence activated cell sorting. MICPB cells may be also isolated on the basis of other characteristics and properties of the cells such as cell size and morphology. Methods for isolation and expansion of MICPB cells are disclosed herein. A method of isolation of MICPB cells consist of obtaining cell population from living donors. A biological sample may be partially purified after collection by removing fat, erythrocytes and plasma by any technique known in the art. Though not bound by theory it is believed that MICPB cells will respond to signals produced by one or more cell types within a biological compartment into which MICPB cells are placed. Thus the cell may be made to differentiate into a number of phenotypically defined cells such as chondroblast, osteoblast, myocytes, defined function fibroblast, nerve cells and other specific cell types. Once a biologic sample is collected and prepared as applicable the sample is further selected and expanded in culture medium. If enough MICPB cells are collected from a single donor, expansion may be avoided. The culture medium can be any cell culture medium or a balanced salt solution that can maintain viable cells at least temporarily. However, none of the available media, at present, can maintain viable cells for extended time periods without being supplemental either with serum or growth factors. Specifically, non-limiting examples of serum are fetal bovine serum, calf serum, horse serum or human serum. The medium can be between about 2% by volume to about 15% by volume of serum. In one embodiment, cell culture medium contains one or more additions such as growth factors, fibronectin coated substrate, antibiotics, and fetal bovine serum. In one embodiment, a method is disclosed for differentiating MICPB cells into chondrogenic cells, which can form cartilage. Chondrogenic differentiation is achieved by culturing MICPB cells in chondrogenic medium. Chondrogenic medium can be any medium or any balanced salt solution that can maintain viable cells and induce chondrogenic differentiation in these cells. Several culture media known in the art can be used for this purpose. In general chondrogenic medium is serum free minimal essential medium. In one embodiment medium is DMEM-high glucose (DMEM-high glucose, Gibco-BR-L) medium. A chondrogenic medium, as an example without limitation, contains specific additives. These may be, but are not limited to TGF beta growth factor, dexamethasone, ascorbic acid, insulin, transferrin, proline, and antibiotics. Glucose levels vary. A method is disclosed herein for differentiating MICPB cells into osteogenic cells. Osteogenic differentiation can be induced by entering MICPB cells in osteogenic medium.

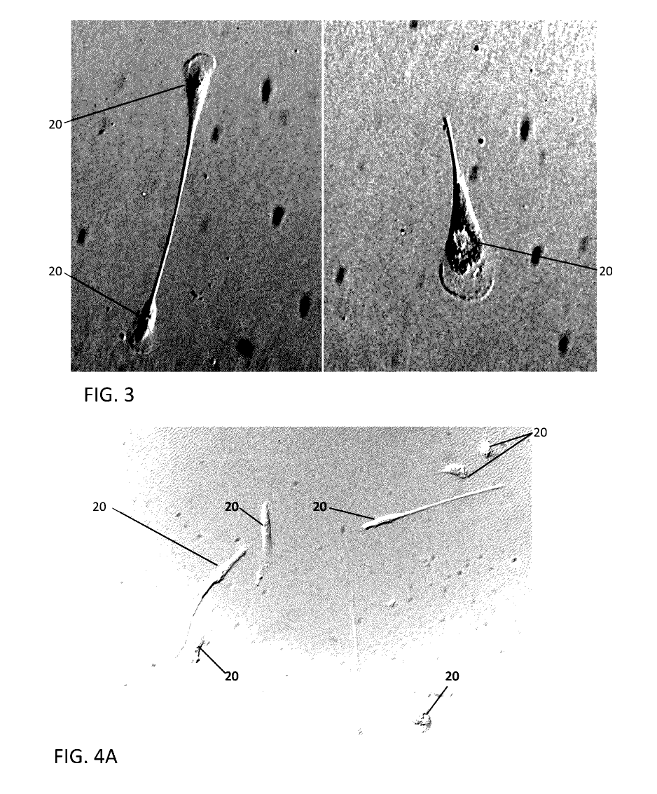

In one exemplary test, the peripheral blood was centrifuged, FICOLL separated and the ring of cells collected from the FICOLL separation was placed in fibronectin coated flasks for selection of stem cells. Seven days after that plating occurred, a flow cytometry analysis was done of those cells. On the second day, the actual markers showing elevated HLADR and CD45 which are hematopoietic cells and immune reactive cells were noted. Those cells were trypsinized and passed to 3 more flasks and they were then allowed to expand. FIG. 3 shows a representative cells isolated from the peripheral blood after 14 days in tissue culture. The cell has the morphologic appearance of primitive mesenchymal cells, similar to those isolated from bone marrow. Hoffman modulation microscopy ×100. The picture was taken 14 days after the passage. This is representative of the cells 20 that were seen. Following that, histograms were obtained from gradient separated blood from a single donor after 30 days incubation, as illustrated in FIGS. 1 and 2. There was no CD15 which is a granulocyte marker. There was no CD73, HLADR, CD29, CD105, SSEA4 or CD34. CD73 is a stem cell marker. HLADR is an immune cell marker. CD29 is a stem cell marker. CD105 is a mesenchymal stem cell marker. SSEA4 is a very primitive stem cell marker. CD34 is a hematopoietic stem cell marker. Testing did show high levels of CD90 which is commonly used as a mesenchymal stem cell marker and nominal levels of CD45 which is a hematopoietic progenitor marker although mesenchymal stem cells have been shown to express low levels of CD45.

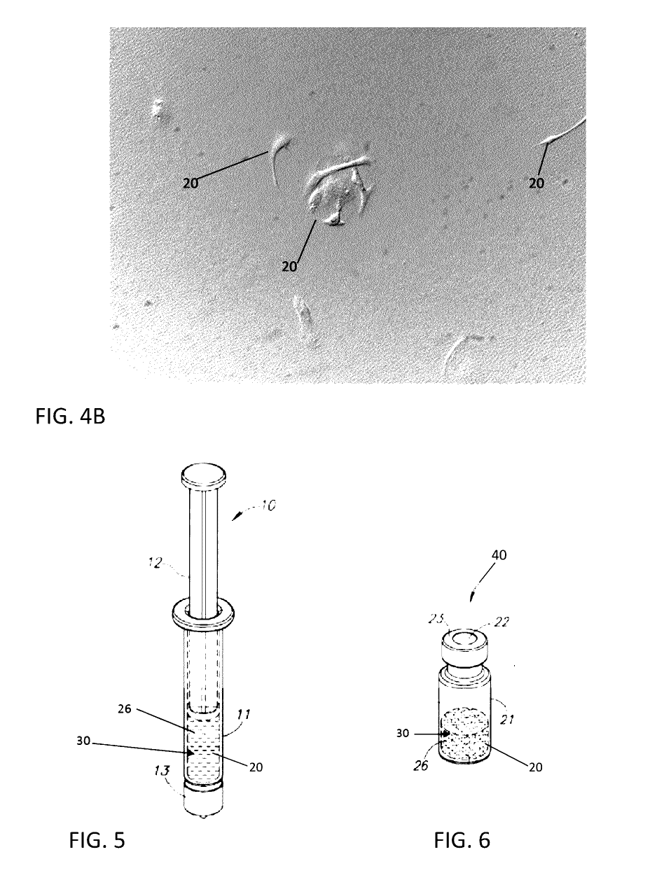

In order to clarify, CD90 is a marker for mesenchymal stem cells and hematopoietic stem cells. Hematopoietic stem cells that are CD90 positive are always CD34 positive, but this cell population has no CD34 in the 30 day flow data showing. FIGS. 4A and 4B show an elongated cell 20 (4A) and a cluster of cells 20 (4B) isolated from peripheral blood after 30 days incubation. These cells 20 showed high levels of CD90 markers. Hoffman modulation microscopy images ×40.

As shown in FIGS. 5 and 6, the composition or mixture 30 of cells 20 is shown is a pre-filled syringe 10 and a sealed container or vial 40, respectively. The composition or mixture 30 has the cells 20 dispersed uniformly in a medium or liquid 26. In either delivery system, the composition 30 can also be filled with saline or other suitable liquid or gel to facilitate a repair or treatment dosage.

As shown in FIG. 5, the syringe 10 has a plunger 12 sealed inside the housing 11 closed by a removable cap 13. The plunger 12 withdrawn to provide volumetric space for the pre-filled mixture 30. Once the cap 13 is removed, a nozzle is exposed to deliver the mixture 30 to the target area to be treated.

Alternatively, as shown in FIG. 6, the entire composition or mixture 30 can be provided in a sealed vial or container 40. An end cap 23 encircles an injectable elastomeric stopper 22 at one end or top of a housing 21. The cells 20 are inside the container housing.

The present invention may be best used as a kit wherein the composition is stored in a sterile vial, a container or a syringe to later use in treating a patient as illustrated in FIGS. 5 and 6. The isolated cells in such a kit may be cultured and expanded to insure a sufficient quantity for therapeutically effective treatments. The cells may even be treated to induce differentiation. In one example, the cells can be induced to osteogenic differentiate by culturing in a medium including a composition of isolated, post-natal, multilineage inducible, morphologically distinct cells from the peripheral blood which express at least CD90.

A method of inducing osteogenic differentiation of a composition has the steps of culturing of the multilineage induced cells in a chondrogenic medium including TBF beta 3, ascorbic acid, dexamethasone, sodium pyruvate, insulin, transferrin, glucose and B-glycerophosphate. The cells can be grown on a thin bone plate with or without perforation. The cells can be grown on decalcified thin bone plate. The cells can be contacted with freeze-dried, dried, dehydrated, frozen, cryopreserved or fresh particulate cartilage to induce chondrogenic differentiation. The cells can be contacted with dried, freeze-dried, dehydrated, frozen, cryopreserved or fresh particulate bone to induce osteogenic differentiation. Also, the cells can be contacted with freeze-dried, dried, dehydrated, frozen, or fresh particulate periosteum to induce osteogenic differentiation. Alternatively, the cells can be contacted with freeze-dried, dried, dehydrated, frozen, cryopreserved or fresh particulate endosteum to effect osteogenic differentiation. The cells also can be placed in contact with freeze-dried, dried, dehydrated, frozen, cryopreserved or fresh particulate nervous tissue derived from the spinal cord, peripheral nerves or brain to effect neural differentiation.

In a preferred method of treating a patient-donor, the composition can be prepared using a method of treating a patient-donor by using a therapeutically effective amount of a composition of isolated, post-natal, multilineage inducible, morphologically distinct cells from the peripheral blood which express at least CD90. This method has the steps of extracting peripheral blood from the patient-donor to be treated to acquire a biologic sample of peripheral blood from which cells are to be isolated; isolating the cells from the peripheral blood which express at least CD90 marker; acquiring a therapeutically effective amount of said cells for treating the patient-donor; and treating said patient-donor with his or her isolated cells taken from the biologic sample. The cells can be grown on a thin bone plate with or without perforation. The cells can be grown on decalcified thin bone plate. The cells can be contacted with freeze-dried, dried, dehydrated, frozen, cryopreserved or fresh particulate cartilage to induce chondrogenic differentiation. The cells can be contacted with dried, freeze-dried, dehydrated, frozen, cryopreserved or fresh particulate bone to induce osteogenic differentiation. The cells can be contacted with freeze-dried, dried, dehydrated, frozen, or fresh particulate periosteum to induce osteogenic differentiation. The cells can be contacted with freeze-dried, dried, dehydrated, frozen, cryopreserved or fresh particulate endosteum to effect osteogenic differentiation. The cells can be placed in contact with freeze-dried, dried, dehydrated, frozen, cryopreserved or fresh particulate nervous tissue derived from the spinal cord, peripheral nerves or brain to effect neural differentiation.

It is understood the patient-donor method provides a most unique way of allowing a patient's own cells to be isolated for later treatments for various conditions and provides the opportunity to establish a replenishable supply of such cells for future use by providing ways to store the material.

Alternatively, the source of such cells can be from cadavers or animal peripheral blood for use in such treatments or other living non-related or related human blood donors.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

1. A composition of isolated, post-natal, multilineage inducible, morphologically distinct cells from the peripheral blood which express at least CD90.

2. The composition of claim 1 wherein the cells are isolated from a biological sample of the peripheral blood, human or animal.

3. The composition of claim 1 wherein the cells do not express markers comprising HLADR and CD34 or high levels of CD45.

4. The composition of claim 1 provided in a kit containing a defined and purified population of multilineage-inducible cells, the kit comprising:
a vial or container of the cells; and
a syringe for removing the cells from the vial or container for delivering the cells to a patient.

5. A method of isolating post-natal, multilineage inducible, morphologically distinct cells from the peripheral blood which express at least CD90 inducing osteogenic or chondrogenic differentiation of a composition, comprising the steps of culturing of the multilineage induced cells in a chondrogenic medium including TBF beta 3, ascorbic acid, dexamethasone, sodium pyruvate, insulin, transferrin and glucose or in osteogenic medium.

6. The method of claim 5 wherein the cells are grown on a thin bone plate with or without perforation.

7. The method of claim 6 wherein cells are grown on decalcified thin bone plate.

8. The method of claim 5 wherein the cells are contacted with freeze-dried, dried, dehydrated, frozen, cryopreserved or fresh particulate cartilage to induce chondrogenic differentiation.

9. The method of claim 5 wherein the cells are contacted with dried, freeze-dried, dehydrated, frozen, cryopreserved or fresh particulate bone to induce osteogenic differentiation.

10. The method of claim 5 wherein cells are contacted with freeze-dried, dried, dehydrated, frozen, or fresh particulate periosteum to induce osteogenic differentiation.

11. The method of claim 5 wherein cells are contacted with freeze-dried, dried, dehydrated, frozen, cryopreserved or fresh particulate endosteum to effect osteogenic differentiation.

12. The method of claim 5 wherein cells are placed in contact with freeze-dried, dried, dehydrated, frozen, cryopreserved or fresh particulate nervous tissue derived from the spinal cord, peripheral nerves or brain to effect neural differentiation.

13. A method of treating a human patient-donor with cells from his or her own peripheral blood using a therapeutically effective amount of a composition of isolated, post-natal, multilineage inducible, morphologically distinct cells from the peripheral blood which express at least CD90 comprising the steps of:
extracting peripheral blood from the patient-donor to be treated;
isolating the cells from the peripheral blood which express at least CD90 marker;
acquiring a therapeutically effective amount of said cells for treating the patient-donor; and
treating said patient-donor with his or her isolated cells taken from the peripheral blood wherein said patient-donor has a nerve or orthopaedic bone or cartilage or connective tissue condition that is treated by the administration of the patient-donor isolated cells to induce repair of said condition.

14. (canceled)

15. (canceled)

16. The method of claim 13 wherein the cells are contacted with freeze-dried, dried, dehydrated, frozen, cryopreserved or fresh particulate cartilage to induce chondrogenic differentiation.

17. The method of claim 13 wherein the cells are contacted with, freeze-dried, dehydrated, cryopreserved or fresh particulate bone to induce osteogenic differentiation.

18. The method of claim 13 wherein cells are contacted with freeze-dried, dried, dehydrated, frozen, or fresh particulate periosteum to induce osteogenic differentiation.

19. The method of claim 13 wherein cells are contacted with freeze-dried, dried, dehydrated, frozen, cryopreserved or fresh particulate endosteum to effect osteogenic differentiation.

20. The method of claim 13 wherein cells are placed in contact with freeze-dried, dried, dehydrated, frozen, cryopreserved or fresh particulate nervous tissue derived from the spinal cord, peripheral nerves or brain to effect neural differentiation.
